SEQUENCE LISTING (3 sequences)

SEQ ID NO: 1 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 2 - protein, 176 aa, Mus musculus
WTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQ

SEQ ID NO: 3 - protein, 377 aa, Mus musculus
MAKSHLLQWLLLLPTLCCPGAAITSASSLECAQGPQFWCQSLEHAVQCRALGHCLQEVWGHAGANDLCQECEDIVHLLTKMTKEDAFQEAIRKFLEQECDILPLKLLVPRCRQVLDVYLPLVIDYFQSQINPKAICNHVGLCPRGQAKPEQNPGMPDAVPNPLLDKLVLPVLPGALLARPGPHTQDFSEQQLPIPLPFCWLCRTLIKRVQAVIPKGVLAVAVSQVCHVVPLVVGGICQCLAERYTVLLLDALLGRVVPQLVCGLVLRCSTEDAMGPALPAVEPLIEEWPLQDTECHFCKSVINQAWNTSEQAMPQAMHQACLRFWLDRQKCEQFVEQHMPQLLALVPRSQDAHITCQALGVCEAPASPLQCFQTPHL

(19) United States
(12) Patent Application Publication
(10) Pub. No.: US 2016/0060329 A1
(43) Pub. Date: Mar. 3, 2016

(54) COMPOSITIONS AND METHODS FOR THE DIAGNOSIS AND PROGNOSIS OF LUNG CANCER

(71) Applicants: Donald SIN, Vancouver (CA); Ayumu TAGUCHI, Houston, TX (US); Stephen LAM, Vancouver (CA); Samir HANASH, Houston, TX (US); Xiaobo DUAN, Victoria (CA); Carl Martin TAMMEMAGI, St. Catharines (CA); Heidi Jo AUMAN, San Francisco, CA (US); Frederica PERERA, New York, NY (US); Brad NELSON, Victoria (CA)

(72) Inventors: Donald SIN, Vancouver (CA); Ayumu TAGUCHI, Houston, TX (US); Stephen LAM, Vancouver (CA); Samir HANASH, Houston, TX (US); Xiaobo DUAN, Victoria (CA); Carl Martin TAMMEMAGI, St. Catharines (CA); Heidi Jo AUMAN, San Francisco, CA (US); Frederica PERERA, New York, NY (US); Brad NELSON, Victoria (CA)

(21) Appl. No.: 14/811,372

(22) Filed: Jul. 28, 2015

Related U.S. Application Data
(60) Provisional application No. 62/030,044, filed on Jul. 28, 2014.

(51) Int. Cl. C07K 16/18; G01N 33/577; G01N 33/574

(52) U.S. Cl. CPC C07K 16/18; G01N 33/57423; G01N 33/577

(57) ABSTRACT

The present invention relates to methods and compositions for the detection of lung cancer. More particularly, the present invention provides monoclonal antibodies for the detection of lung cancer.

### FIELD OF INVENTION

The present invention relates to methods and compositions for the detection of lung cancer. More particularly, the present invention provides monoclonal antibodies for the detection of lung cancer.

### BACKGROUND OF THE INVENTION

Despite reduced smoking rates in the western world, lung cancer remains the leading cause of cancer mortality in the US and elsewhere. In 2013, it was projected that over 160,000 Americans would die from lung cancer, which represents 29% of all cancer deaths in men and 26% of all cancer deaths in women.¹ Lung cancer survival is largely dependent on stage at diagnosis. Whereas localized disease (without lymphatic or distant spread) is associated with a 5 year survival greater than 50%, those with distant or regional metastasis have survival measured in weeks to months.¹ Unfortunately, less than 15% of all tumors are found as localized disease. The advent and widespread availability of thoracic computed tomography (CT) scanning has the potential to shift detection to earlier stages and thus improve survival. Data from the National Lung Screening Trial (NLST) suggest that yearly screening with low-dose thoracic CT scan in high-risk current and ex-smokers reduces lung cancer mortality by 20% and total mortality by 7%.² However, if these data are generalized and applied to the entire US population, CT screening strategy would cost $1.3 to $2 billion per year.³ Selection of individuals for lung cancer screening based on high risk rather than the NLST criteria (age 55-79 years, ≧30 pack-years smoked, <15 years quit-time) has been shown to save more lives and to be more efficient.⁹

Surfactant protein B (SFTPB) is synthesized initially as a hydrophilic 42 kiloDalton (kD) protein (pro-SFTPB) by type 2 alveolar pneumocytes and nonciliated bronchiolar cells. Upon synthesis, pro-SFTPB quickly undergoes proteolytic cleavage by cysteine proteases in the endoplastic reticulum resulting in the synthesis and secretion of a 9 kD non-collagenous hydrophobic SFTPB, which is the functional mature form of SFTPB.⁴ Lung tumor cells (such as adenocarcinomas) may exhibit dysregulated SFTPB synthesis, leading to the over-expression of pro-SFTPB with muted ability to post-translationally modify the precursor into the mature hydrophobic form.^{5,6} In one study, increased levels of circulating mature SFTPB were found in subjects with resectable NSCLC relative to matched controls.⁷

### SUMMARY OF THE INVENTION

The present invention provides, in part, methods and compositions for the detection of lung cancer.

In one aspect, the invention provides a monoclonal antibody, or an antigen-binding fragment thereof, that specifically binds the N-terminal propeptide of surfactant protein B (NT pro-SFTPB) or fragment thereof, or to a sequence substantially identical to the sequence of NT pro-SFTPB or fragment thereof.

In some embodiments, the monoclonal antibody does not significantly bind one or more of mature surfactant protein B, the signal peptide of surfactant protein B, or the C-terminal propeptide of surfactant protein B.

In some embodiments, the pro-SFTPB may be human pro-SFTPB. In some embodiments, the pro-SFTPB may essentially have the amino acid sequence as set forth in SEQ ID NO: 2, or a fragment thereof.

In some embodiments, the monoclonal antibody may be linked to a detectable label, such as biotin.

In some embodiments, the monoclonal antibody may be linked to a solid support.

In some aspects, the invention provides a hybridoma cell line producing a monoclonal antibody as described herein, such as clones ACcSFTPB.3409 or ACcSFTPB.3473.

In some aspects, the invention provides a composition including an antibody as described herein, and at least one of a physiologically acceptable carrier, diluent, excipient, or stabilizer.

In some aspects, the invention provides a method for detecting the N-terminal propeptide of surfactant protein B (NT pro-SFTPB) in a biological sample, by contacting the biological sample with a monoclonal antibody as described herein under conditions such that the antibody binds to the NT pro-SFTPB, if present in the biological sample; and detecting the presence, absence, or amount of binding of the antibody to the NT pro-SFTPB from the biological sample. In some embodiments, the monoclonal antibody may be linked to a solid support. In some embodiments, after the contacting, unbound components of the sample may be washed away from the monoclonal antibody linked to the solid support while the NT pro-SFTPB if present, remains bound to the monoclonal antibody, and the NT pro-SFTPB bound to the monoclonal antibody linked to the solid support may be contacted with a second monoclonal antibody that binds the NT pro-SFTPB and the presence, absence, or amount of the second monoclonal antibody may be detected. In some embodiments, the monoclonal antibody or the second monoclonal antibody may be linked to a detectable label.

In some aspects, the invention provides a kit including a monoclonal antibody as described herein, together with instructions for detecting the N-terminal propeptide of surfactant protein B (NT pro-SFTPB) in a biological sample.

In some embodiments, the biological sample is a biological fluid, such as whole blood or plasma.

In some aspects, the invention provides a method of diagnosing or prognosing lung cancer in a subject, by detecting the presence or absence of the N-terminal propeptide of surfactant protein B (NT pro-SFTPB), where the presence of NT pro-SFTPB may be a diagnosis or prognosis of lung cancer in the subject. The lung cancer may be non-small cell lung cancer (NSCLC), lung adenocarcinoma or lung squamous cell carcinoma. The subject may be a human.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 is a flow diagram of subject recruitment into the Pan-Canadian Study;

FIGS. 2A-B show the schema of mouse and human SFTPB and mass spectrometric identification of SFTPB peptides. FIG. 2A. Schema of mouse surfactant protein B. Plasma samples from three mouse models of lung adenocarcinoma (EGFR; TetO-EGFR^{L858R}/CCSP-rtTA, Kras; TetO-Kras4b^{G12D}/CCSP-rtTA, and Urethane; urethane treated) were analyzed by mass spectrometry previously. Gray bars indicate peptides identified in plasma of each lung adenocarcinoma mouse model. All presented amino acid positions are based on P50405 in UniProtKB. FIG. 2B. Schema of human SFTPB and identification of peptides by mass spectrometry in the conditioned media of human NSCLC cell lines. Potential SFTPB isoforms were identified in the conditioned media of H3255 and HCC4019. Bars indicate potential SFTPB isoforms and gray regions in the bars indicate identified peptides in the same protein fraction. Numbers indicate the sum of the number of mass spectra counts for each peptide. A black bar indicates the peptide used as an immunogen to develop monoclonal antibodies. All presented amino acid positions are based on P07988 in UniProtKB;

FIGS. 3A-B are graphs showing plasma pro-SFTPB levels in newly diagnosed NSCLC set. FIG. 3A. Levels of pro-SFTPB in plasmas from newly diagnosed NSCLC subjects and from non-cancer controls. Columns indicate 25th and 75th percentiles, horizontal lines in columns indicate median, bars indicate 10th and 90th percentiles, and black dots indicate data outside the 10th and 90th percentiles. FIG. 3B. ROC analysis of pro-SFTPB and mature SFTPB. Mature SFTPB was assayed previously. AUC, area under the curve;

FIG. 4 is a graph showing the sensitivity and specificity for the unadjusted logistic regression model of log-transformed pro-SFTPB predicting lung cancer in the Pan-Canadian Early Detection of Lung Cancer Study. Abbreviations: pro-SFTPB, pro-surfactant protein B;

FIG. 5 is a graph showing receiver operator characteristic curves for the full model with and without pro-SFTPB in the Pan-Canadian Early Detection of Lung Cancer. Abbreviations: AUC, receiver operator characteristic area under the curve. SFTPB, surfactant protein B.

FIG. 6 is a graph showing the mean absolute error (observed—predicted probabilities) for prediction models with (Table 3) and without log-transformed pro-SFTPB. Abbreviations: pro-SFTPB, pro-surfactant protein B. Circles: With Pro-SFTPB; Triangles: Without Pro-SFTPB; and

FIG. 7 is a graph showing ROC analysis of pro-SFTPB. AUC, area under the curve.

### DETAILED DESCRIPTION

The present disclosure provides, in part, methods and compositions for the detection of lung cancer.

Surfactant protein B (SFTPB) is synthesized initially as a hydrophilic 42 kiloDalton (kD) protein (pro-SFTPB) by type 2 alveolar pneumocytes and nonciliated bronchiolar cells. Upon synthesis, pro-SFTPB quickly undergoes proteolytic cleavage by cysteine proteases in the endoplastic reticulum, releasing a signal peptide and N- and C-terminal pro-peptides, and resulting in the synthesis and secretion of a 9 kD non-collagenous hydrophobic SFTPB, which is the functional mature form of SFTPB.

In some embodiments, the SFTPB may be human SFTPB or may be mouse SFTPB.

In some embodiments, the human SFTPB may have the sequence set forth in UniProtKB/Swiss-Prot entry P07988:

(SEQ ID NO: 1)
MAESHLLQWLLLLLPTLCGPGTAAWTTSSLACAQGPEFWCQSLEQA
LQCRALGHCLQEVWGHVGADDLCQECEDIVHILNKMAKEAIFQDTM
RKFLEQECNVLPLKLLMPQCNQVLDDYFPLVIDYFQNQTDSNGICM
HLGLCKSRQPEPEQEPGMSDPLPKPLRDPLPDPLLDKLVLPVLPGA
LQARPGPHTQDLSEQQFPIPLPYCWLCRALIKRIQAMIPKGALAVA
VAQVCRVVPLVAGGICQCLAERYSVILLDTLLGRMLPQLVCRLVLR
CSMDDSAGPRSPTGEWLPRDSECHLCMSVTTQAGNSSEQAIPQAML
QACVGSWLDREKCKQFVEQHTPQLLTLVPRGWDAHTTCQALGVCGT
MSSPLQCIHSPDL,

in which residues 1-24 form the signal peptide, 25-200 form the N-terminal pro-peptide, 201-279 form the mature pulmonary surfactant-associated protein B, and 280-381 form the C-terminal pro-peptide.

In some embodiments, the mouse N-terminal pro-peptide (25-200 aa) may have the following sequence:

(SEQ ID NO: 2)
WTTSSLACAQGPEFWCQSLEQALQCRALGHCLQEVWGHVGADDLCQ
ECEDIVHILNKMAKEAIFQDTMRKFLEQECNVLPLKLLMPQCNQVL
DDYFPLVIDYFQNQTDSNGICMHLGLCKSRQPEPEQEPGMSDPLPK
PLRDPLPDPLLDKLVLPVLPGALQARPGPHTQDLSEQQ.

In some embodiments, the mouse SFTPB may have the sequence set forth in P50405 in UniProtKB:

(SEQ ID NO: 3)
MAKSHLLQWLLLLPTLCCPGAAITSASSLECAQGPQFWCQSLEHAV
QCRALGHCLQEVWGHAGANDLCQECEDIVHLLTKMTKEDAFQEAIR
KFLEQECDILPLKLLVPRCRQVLDVYLPLVIDYFQSQINPKAICNH
VGLCPRGQAKPEQNPGMPDAVPNPLLDKLVLPVLPGALLARPGPHT
QDFSEQQLPIPLPFCWLCRTLIKRVQAVIPKGVLAVAVSQVCHVVP
LVVGGICQCLAERYTVLLLDALLGRVVPQLVCGLVLRCSTEDAMGP
ALPAVEPLIEEWPLQDTECHFCKSVINQAWNTSEQAMPQAMHQACL
RFWLDRQKCEQFVEQHMPQLLALVPRSQDAHITCQALGVCEAPASP
LQCFQTPHL,

in which residues 1-22 form the signal peptide, 23-191 form the N-terminal pro-peptide, 192-270 form the mature pulmonary surfactant-associated protein B, and 271-377 form the C-terminal pro-peptide.

In some embodiments, the mouse N-terminal pro-peptide (23-191 aa) may have the following sequence:

(SEQ ID NO: 4)
ITSASSLECAQGPQFWCQSLEHAVQCRALGHCLQEVWGHAGANDLC
QECEDIVHLLTKMTKEDAFQEAIRKFLEQECDILPLKLLVPRCRQV
LDVYLPLVIDYFQSQINPKAICNHVGLCPRGQAKPEQNPGMPDAVP
NPLLDKLVLPVLPGALLARPGPHTQDFSEQQ.

SFTPB fragments may include, without limitation, any antigenic fragment. In to some embodiments, SFTPB fragments may include, without limitation, fragments identified by mass spectrometry, as described herein.

In one aspect, the present disclosure provides a monoclonal antibody, or an antigen-binding fragment thereof, that specifically binds the N-terminal propeptide of surfactant protein B (NT pro-SFTPB) or fragment thereof, or to a sequence substantially identical to the sequence of NT pro-SFTPB or fragment thereof. In some embodiments, the monoclonal antibody does not substantially recognise and bind one or more of mature surfactant protein B, the signal peptide of surfactant protein B, or the C-terminal propeptide of surfactant protein B.

By “substantially identical” is meant an amino acid or nucleic acid sequence exhibiting at least 90%, for example, 90%, 91%, 92%, 93%, 94%, 95%, 96%, 97%, 98%, or 99% sequence identity to a reference polypeptide sequence, such as a NT pro-SFTPB, or nucleic acid encoding a NT pro-SFTPB. The term “identity” shall be construed to mean the percentage of amino acid or nucleic acid residues in the candidate sequence that are identical with the residue of a corresponding sequence to which it is compared, after aligning the sequences and introducing gaps, if necessary, to achieve the maximum percent identity for the entire sequence, and not considering any conservative substitutions as part of the sequence identity. Neither N- or C-terminal extensions nor insertions should be construed as reducing identity or homology. Methods and computer programs for the alignment are well known in the art. Sequence identity may be measured using sequence analysis software.

By “antibody” is meant a protein that specifically binds an antigen, including without limitation polyclonal antibodies, monoclonal antibodies, chimeric antibodies, humanized antibodies, human antibodies or antigen-binding fragments thereof. Antibodies are generally tetrameric molecules, having two identical heavy (H) chains and two identical light (L) chains. Each heavy and light chain contains a variable domain (V_{H} or V_{L}, respectively) at its N-terminus, followed by several constant domains Antigen-binding fragments may include without limitation Fab, Fab′, F(ab′)₂ and Fv fragments. By “epitope” or “antigenic determinant” is meant the amino acids to which an antibody binds. The amino acids may be a contiguous amino acid sequence, or may be noncontiguous amino acids that form the epitope due to the tertiary structure of the antigen. An antibody “specifically binds” an antigen when it recognises and binds the antigen, for example, NT pro-SFTPB, but does not substantially recognise and bind other molecules in a sample, for example, a mature surfactant protein B, a signal peptide of surfactant protein B, a C-terminal propeptide of surfactant protein B, or other surfactant or lung-expressed protein, or fragment thereof. Such an antibody may have, for example, an affinity for the antigen which is at least 10, 100, 1000 or 10000 times greater than the affinity of the antibody for the other molecules in the sample.

By “monoclonal antibody” is meant an antibody produced by clonal antibody-producing cell such as a hybridoma, lymphocyte, or a recombinant antibody-producing cell. Monoclonal antibodies are directed to a single epitope or antigenic determinant and can be prepared using standard techniques. For example, a hybridoma can be prepared by immunizing a host animal, such as a mouse, rat, hamster, or rabbit, with an antigen (for example, NT pro-SFTPB) to generate lymphocytes that are capable of producing antibodies that will specifically bind to the antigen. Lymphocytes obtained from the immunized host animal can then be fused with myeloma or other tumor cells to generate hybridoma cells capable of repeated cell divisions. Clones, such as clones ACcSFTPB.3409 and ACcSFTPB.3473 described herein, can be selected by any suitable means, for example, dilution or single-cell selection.

It is to be understood that alternative methods of producing monoclonal antibodies, including human monoclonal antibodies, are known in the art, and any suitable method may be used. In some embodiments, antibodies according to the present disclosure can be “substantially pure” or “isolated,” for example, separated from hybridoma or other cells or cellular components. In some embodiments, monoclonal antibodies according to the present disclosure may constitute at least 90, 95, or 99% of all protein in a solution.

In some embodiments, the antibody, such as a monoclonal antibody, may be linked to a detectable label. By “detectable label” is meant a molecule that can be directly or indirectly conjugated to the antibody, for marking and identifying the presence of the antibody by, for example, spectroscopic, photochemical, biochemical, immunochemical, optical, chemical, or physical means. For example, the label can be directly attached to the antibody or to another agent, such as a secondary antibody. Any suitable label can be used, as long as it does not significantly interfere with the specific binding of the antibody to its antigen and permits detection of the antibody. Methods for detectably-labelling a molecule are well known in the art and include, without limitation, radioactive labelling (e.g., with an isotope such as ³²P or ³⁵S) and nonradioactive labelling such as, enzymatic labelling (for example, using horseradish peroxidase, alkaline phosphatase, or other enzymes used in, for example, ELISA), chemiluminescent labeling, fluorescent labeling (for example, using fluorescein, Texas red, rhodamine, etc.), bioluminescent labeling, or antibody detection of a ligand attached to the probe. Also included in this definition is a molecule that is detectably labeled by an indirect means, for example, a molecule that is bound with a first moiety (such as biotin) that is, in turn, bound to a second moiety that may be observed or assayed (such as streptavidin). Labels can also include digoxigenin, luciferases, or aequorin.

In some embodiments, the monoclonal antibody may be attached or linked to a solid support. By “solid support” is meant any non-aqueous matrix, which is chemically inert and insoluble in an assay solution, to which a molecule, such as an antibody, can adhere or be conjugated. Any suitable solid support can be used, such as beads, microparticles, glass, polymers such as polysaccharides (e.g., agarose), polyacrylamides, polystyrene, polyvinyl alcohol, silicones, magnetic or chromatographic matrix particles, the surface of an assay plate (e.g., microtiter wells), pieces of a solid substrate material or membrane (e.g., plastic, nylon, paper), etc. In some embodiments, the solid support can be the interior of an assay container, such as the well of an assay plate; a dipstick; a particle inside an assay container, etc. The attachment or linkage of the antibody to the solid support can be by any suitable means, such as by electrostatic attraction, affinity interaction, hydrophobic interaction, covalent bonding, etc.

In some embodiments, immunological techniques can be used to detect the presence, absence, or level of SFTPB, such as NT pro-SFTPB, in a sample. Such techniques can include, without limitation, enzyme immunoassays (EIA) such as enzyme multiplied immunoassay technique (EMIT), enzyme-linked immunosorbent assay (ELISA), antigen capture ELISA, sandwich ELISA, IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); or chemiluminescence assays (CL).

In some embodiments, antigen capture ELISA can be used to detect the presence or level of pro-SFTPB in a sample. For example, an antibody directed to pro-SFTPB can be linked to a solid support and sample can be added such that pro-SFTPB, if present, is bound by the antibody. After unbound proteins are removed by washing, the amount of bound marker can be quantified by for example a radioimmunoassay, using standard techniques.

In some embodiments, sandwich ELISA can be used to detect pro-SFTPB in a sample. For example, in a two-antibody sandwich assay, a first (capture) antibody can be bound to a solid support, and pro-SFTPB, if present, can be allowed to bind to the first antibody. Other components of the sample can be optionally removed (e.g., washed away) before a second (detection) antibody is contacted to the antigen bound to the capture antibody. The amount of the marker is quantified by measuring the amount of a second (capture) antibody that binds pro-SFTPB. The antibodies can be immobilized onto a variety of solid supports, as described herein. In some embodiments, an assay strip can be prepared by coating the antibody or a plurality of antibodies in an array on a solid support. This strip can then be dipped into the test sample and processed quickly through washes and detection steps to generate a measurable signal, such as a colored spot. In some embodiments, the capture antibody can be the antibody produced by clone ACcSFTPB.3473 (antibody 515) and the detection antibody can be the antibody produced by clone ACcSFTPB.3409 (antibody 477).

In some embodiments, specific immunological binding of the antibody to pro-SFTPB can be detected directly or indirectly. Direct detectable labels may include fluorescent or luminescent tags, metals, dyes, radionuclides, etc., which can be attached to the antibody. An antibody labeled with iodine-125 (¹²⁵I), for example, can be used for determining the level of pro-SFTPB in a sample. A chemiluminescence assay using a chemiluminescent antibody specific for pro-SFTPB may be suitable for sensitive, non-radioactive detection of pro-SFTPB levels. An antibody labeled with fluorochrome may also be suitable for determining the levels of pro-SFTPB in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially.

Indirect labels may include, without limitation, horseradish peroxidase (HRP), alkaline phosphatase (AP), β-galactosidase, urease, etc. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used, for example, with the chromogenic substrate p-nitrophenyl phosphate, which yields a soluble product readily detectable at 405 nm. Similarly, a β galactosidase detection system can be used with the chromogenic substrate o-nitrophenyl-β-D-galactopyranoside (ONPG), which yields a soluble product detectable at 410 nm A urease detection system can be used with a substrate such as urea-bromocresol purple (Sigma Immunochemicals; St. Louis, Mo.). Suitable secondary antibodies linked to an enzyme are available from commercial sources.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of marker levels can be made using a spectrophotometer such as an EMAX Microplate Reader (Molecular Devices; Menlo Park, Calif.) in accordance with the manufacturer's instructions. If desired, the assays described herein can be automated or performed robotically, and the signal from multiple samples can be detected simultaneously.

Quantitative Western blotting can also be used to detect or determine the presence or level of pro-SFTPB in a sample. Western blots can be quantified by methods such as scanning densitometry or phosphorimaging. As a non-limiting example, protein samples are electrophoresed on 10% SDS-PAGE Laemmli gels. Murine monoclonal antibodies are reacted with the blot, and antibody binding can be confirmed to be linear using a preliminary slot blot experiment. Goat anti-mouse horseradish peroxidase-coupled antibodies (BioRad) can be used as the secondary antibody, and signal detection performed using chemiluminescence, for example, with the Renaissance chemiluminescence kit (New England Nuclear; Boston, Mass.). The blots can be analyzed using a scanning densitometer (Molecular Dynamics; Sunnyvale, Calif.) and normalized to a positive control. Values can be reported, for example, as a ratio between the actual value to the positive control (densitometric index).

Alternatively, a variety of immunohistochemical assay techniques can be used to detect or determine the presence or level of pro-SFTPB in a sample. The term “immunohistochemical assay” includes, without limitation, techniques that utilize the visual detection of fluorescent dyes or enzymes coupled or conjugated to antibodies that react with pro-SFTPB using fluorescent microscopy or light microscopy (e.g., in a tissue slice) and includes, without limitation, direct fluorescent antibody assay, indirect fluorescent antibody (IFA) assay, anticomplement immunofluorescence, avidin-biotin immunofluorescence, and immunoperoxidase assays. An IFA assay, for example, is useful for determining whether a sample is positive for pro-SFTPB or the level of pro-SFTPB in a sample. The concentration of pro-SFTPB in a sample can be quantified through for example, endpoint titration or measuring the visual intensity of fluorescence compared to a known reference standard.

In some embodiments, pro-SFTPB can be detected as part of a multiplex assay. The analysis of a plurality of markers may be carried out separately or simultaneously with one test sample using, for example, microarray or other techniques known in the art.

The sample can be a biological sample, for example, any organ, tissue, cell, or cell extract isolated from a subject, such as a sample isolated from a mammal having a lung cancer. For example, a sample can include, without limitation, cells or tissue (e.g., from a biopsy or autopsy) from lung, bodily fluid, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, urine, stool, saliva, placental extracts, amniotic fluid, a cryoprecipitate, a cryosupernatant, a cell lysate, mammalian cell culture or culture medium, products of fermentation, ascitic fluid, proteins present in blood cells, or any other specimen or clinical sample, or any extract thereof, obtained from a patient (human or animal), test subject, or experimental animal. In some embodiments, it may be desirable to separate cancerous cells from non-cancerous cells in a sample. A sample may also include, without limitation, products produced in cell culture by normal or transformed cells (e.g., via recombinant DNA or monoclonal antibody technology). A sample may also include, without limitation, any organ, tissue, cell, or cell extract isolated from a non-mammalian subject, such as an insect or a worm. A “sample” may also be a cell or cell line created under experimental conditions, that is not directly isolated from a subject. A sample can also be cell-free, artificially derived or synthesised. A sample may be from a cell or tissue known to be cancerous, suspected of being cancerous, or believed not be cancerous (e.g., normal or control).

A “control” or reference includes a sample obtained for use in determining base-line expression or activity. Accordingly, a control sample may be obtained by a number of means including from non-cancerous cells or tissue e.g., from cells surrounding a tumor or cancerous cells of a subject; from subjects not having a cancer; from subjects not suspected of being at risk for a cancer; or from cells or cell lines derived from such subjects. A control also includes a previously established standard. Accordingly, any test or assay conducted according to the invention may be compared with the established standard and it may not be necessary to obtain a control sample for comparison each time.

In some embodiments, the present disclosure provides kits for performing an immunoassay using one or more (e.g., two) pro-SFTPB antibodies as described herein. In some embodiments, the kit may include a pro-SFTPB antibody as described herein linked to a solid support. In some embodiments, the kit may include a pro-SFTPB as described herein linked to a detectable label. In some embodiments, the kit may include a secondary antibody that binds to the pro-SFTPB detection antibody (such as the antibody in a sandwich assay that is not linked to the solid support). In some embodiments, the kit may include a pro-SFTPB antibody as described herein linked to a solid support and at least one pro-SFTPB as described herein linked to a label. In some embodiments, the antibody linked to the solid support may be the antibody produced by clone ACcSFTPB.3473 (antibody 515; the capture antibody) and the antibody linked to the detectable label may be the antibody produced by clone ACcSFTPB.3409 (antibody 477; the detection antibody). The kits may also include other reagents, such as reagents for using or developing an ELISA assay.

Detection of pro-SFTPB, such as NT pro-SFTPB, may be useful for providing a diagnosis or prognosis of lung cancer, or for monitoring disease progression and/or monitoring treatment of lung cancer in a subject. As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or being at risk of having a lung cancer, be diagnosed with a lung cancer, or be a control subject that is confirmed to not have a lung cancer. In some embodiments, the subjects may be at high risk for lung cancer. In some embodiments, the subjects may have no clinical history of lung cancer. In some embodiments, the subjects may be screened for lung cancer as described herein. Diagnostic methods for lung cancer and the clinical delineation of such diagnoses are known to those of ordinary skill in the art.

In some embodiments, the pro-SFTPB may be circulating (e.g., in blood) pro-SFTPB.

In some embodiments, the detection of pro-SFTPB may be early detection of lung cancer in a subject who is, for example, assessed to be at risk for developing lung cancer according to existing lung cancer risk prediction models as described herein or known in the art. In some embodiments, the detection of pro-SFTPB may be used to augment clinical information in risk-stratifying smokers for early lung cancer detection.

In some embodiments, the lung cancer may be non-small cell lung cancer (NSCLC), such as lung adenocarcinoma, lung large cell carcinoma or lung squamous cell carcinoma. In some embodiments, the NSCLC may be an early staged NSCLC tumor, which may be amenable to surgical resection. In some embodiments, the lung cancer may be small cell lung cancer, such as lung small cell carcinoma, lung mixed small cell/large cell carcinoma or lung combined small cell carcinoma.

In some embodiments, the detection of pro-SFTPB may be conducted separately, in combination with, or in addition to, reagents or antibodies to other biomarkers in, for example, a biomarker panel for early detection, classification, risk assessment, diagnosis or prognosis of lung cancer, such as NSCLC. In some embodiments, the detection of pro-SFTPB may be conducted separately, in combination with, or in addition to, thoracic CT for lung cancer screening.

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

### Example

### Methods

Study Populations

PanCan Study

The initial work was performed on data from the multicenter Pan-Canadian Early Detection of Lung Cancer (PanCan) Study (ClinicalTrials[dot]gov NCT00751660), which enrolled 2,537 individuals free of a prior history of lung cancer but with a minimum 2% 3-year risk of lung cancer as predicted by lung cancer risk prediction models.^{8,9}

The inclusion criteria for the PanCan study were as follows:
Women or men age 50 to 75 years;
Current or former smokers who have smoked cigarettes for 20 years or more (a former smoker is defined as one who has stopped smoking for one or more years);
An estimated 3-year lung cancer risk of 2% based on the risk prediction model;
ECOG performance status 0 or 1;
Capable of providing, informed consent for screening procedures (low dose spiral CT, AFB, spirometry, blood biomarkers).

The exclusion criteria for the PanCan study were as follows:
Any medical condition, such as severe heart disease (e.g. unstable angina, chronic congestive heart failure), acute or chronic respiratory failure, bleeding disorder, that in the opinion of the investigator could jeopardize the subject's safety during participation in the study or unlikely to benefit from screening due to shortened life-expectancy from the co-morbidities;
Diagnosis of cancer except for non-melanomatous skin cancer, localized prostate cancer, carcinoma in situ (CIS) of the cervix, or superficial bladder cancer with the last treatment 5 years or less prior to registration onto this study;
Ex-smoker for 15 years or more;
On Anti-coagulant treatment such as warfarin or heparin;
Known reaction to Xyocaine, salbutamol, midazolam, and alfentanil;
Pregnancy;
Unwilling to have a spiral chest CT;
Unwilling to provide written consent;
Chest CT within 2 years.

Following informed consent, at baseline all participants completed a structured epidemiologic questionnaire and had blood samples drawn and processed and stored in a study biorepository. The subjects also underwent low dose non-contrast enhanced thoracic CT scanning and performed spirometry, according to the American Thoracic Society/European Respiratory Society guidelines.¹⁰ All subjects were followed up in person at least every 6 months for at least 2 years or until the date of lung cancer diagnosis, date of death, loss to follow-up, or Feb. 1, 2013, whichever came first. The primary outcome was the occurrence of lung cancer during follow-up. The study was approved by the Clinical Research Ethics Board of the University of British Columbia and at each of the participating PanCan Study sites.

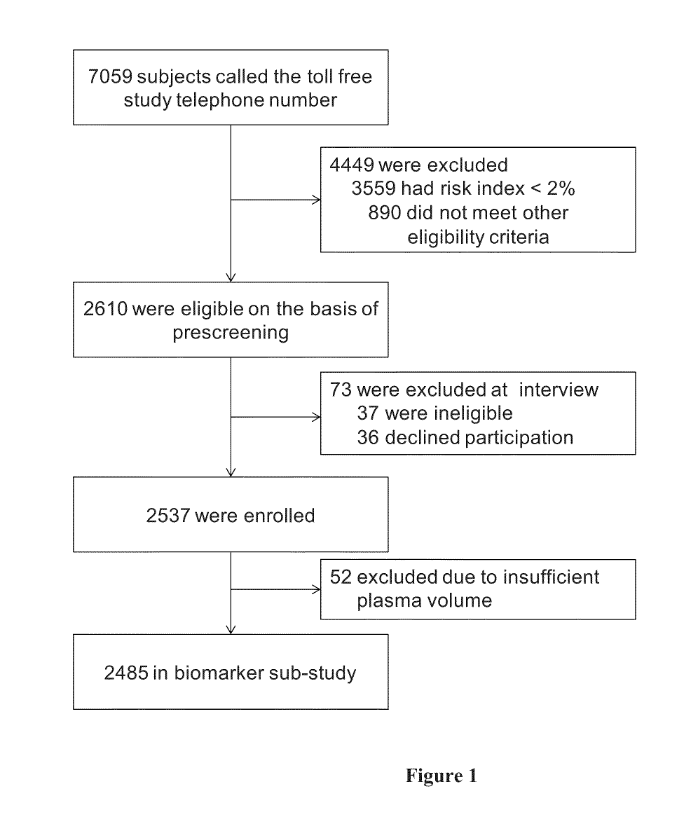

Study enrollment began 24 Sep. 2008 and was completed on 17 Dec. 2010 (FIG. 1). As of 1 Feb. 2013, 113 of 2,537 individuals had been diagnosed with lung cancer. The minimum, median and maximum follow-up durations were 0.02, 3.02 and 4.36 years. During this follow-up period, 187 (7.4%) individuals were lost to follow-up. The overall cumulative incidence of lung cancer was 4.45% and the annual incidence rate of lung cancer was 1.48 per 100 person-years of follow-up. Distributions of study variables by lung cancer status are presented in Table 1.

TABLE 1
Baseline characteristics of Pan-Canadian Early Detection of Lung
Cancer Study participants by lung cancer status and overall

| | Lung cancer | | | |
|---|---|---|---|---|
| | No cancer | Cancer | Total | |
| Characteristic | n (%) | n (%) | N (%) | P-value* |
| Age (years) | | | | |
| 50-54 | 234 (95.90) | 10 (4.10) | 244 (10.38) | |
| 55-59 | 443 (96.72) | 15 (3.28) | 458 (19.49) | |
| 60-64 | 726 (95.53) | 34 (4.47) | 760 (32.34) | |
| 65-69 | 568 (93.57) | 39 (6.43) | 607 (25.83) | |
| ≧70 | 266 (94.66) | 15 (5.34) | 281 (11.96) | 0.036^{†} |
| Sex | | | | |
| Male | 1242 (96.13) | 50 (3.87) | 1292 (54.98) | |
| Female | 995 (94.05) | 63 (5.95) | 1058 (45.02) | 0.020 |
| Body Mass Index (BMI) | | | | |
| Underweight (<18.5 kg/m²) | 20 (95.24) | 1 (4.76) | 21 (0.88) | |
| Normal (18.5-24.9 kg/m²) | 803 (93.59) | 55 (6.41) | 858 (36.51) | |
| Overweight (25-29.9 kg/m²) | 1031 (96.18) | 41 (3.82) | 1072 (45.62) | |
| Obese (≧30 kg/m²) | 383 (95.99) | 16 (4.01) | 399 (16.98) | 0.016^{†} |
| Personal History of Cancer | | | | |
| No | 2095 (95.36) | 102 (4.64) | 2197 (93.77) | |
| Yes | 135 (92.47) | 11 (7.53) | 146 (6.23) | 0.112 |
| Family History of Cancer | | | | |
| No | 1458 (95.54) | 68 (4.46) | 1526 (66.12) | |
| Yes | 740 (94.63) | 42 (5.37) | 782 (33.88) | 0.353 |
| Pneumonia | | | | |
| No | 1648 (95.76) | 73 (4.24) | 1721 (73.42) | |
| Yes | 583 (93.58) | 40 (6.42) | 623 (26.58) | 0.037 |
| Emphysema | | | | |
| No | 2091 (95.264) | 104 (4.74) | 2195 (93.76) | |
| Yes | 137 (93.84) | 9 (6.16) | 146 (6.24) | 0.423 |
| Smoking status | | | | |
| Former smoker | 871 (95.61) | 40 (4.39) | 911 (38.77) | |
| Current smoker | 1366 (94.93) | 73 (5.07) | 1439 (61.23) | 0.489 |
| Race/Ethnicity | | | | |
| White | 2175 (95.14) | 111 (4.86) | 2286 (97.65) | |
| Asian | 22 (95.65) | 1 (4.35) | 23 (0.98) | |
| Aboriginal | 7 (100) | 0 (0) | 7 (0.30) | |
| Black or African Canadian | 12 (92.31) | 1 (7.69) | 13 (0.56) | |
| Other | 12 (100) | 0 (0.00) | 12 (0.51) | 0.674^{†} |
| Education | | | | |
| 8^{th} grade or less | 63 (94.03) | 4 (5.97) | 67 (2.85) | |
| 9^{th} to 11^{th} grade | 288 (94.12) | 18 (5.88) | 306 (13.02) | |
| High school graduate | 585 (96.22) | 23 (3.78) | 608 (25.87) | |
| Technical/Vocational | 239 (94.09) | 15 (5.91) | 254 (10.81) | |
| Ass. degree/some college | 428 (94.48) | 25 (5.52) | 453 (19.28) | |
| Bachelor's degree | 401 (96.63) | 14 (3.37) | 415 (17.66) | |
| Advanced degree | 233 (94.33) | 14 (5.67) | 247 (10.51) | 0.936^{†} |
| FEV₁ % pred | | | | |
| N | 2220 | 111 | 2331 | |
| Mean (SD) | 0.82 (0.18) | 0.77 (0.19) | 0.82 (0.18) | |
| Median (IQR) | 0.83 (0.71-0.94) | 0.77 (0.67-0.86) | 0.83 (0.71-0.94) | |
| Range | 0.15-1.68 | 0.27-1.36 | 0.15-1.68 | 0.0034^{‡} |
| Pro-SFTPB (ng/ml) | | | | |
| N | 2193 | 112 | 2305 | |
| Mean (SD) | 43.5 (42.1) | 75.0 (64.5) | 45.1 (43.9) | |
| Median (IQR) | 30.7 (16.5-54.3) | 54.2 (30.8-99.1) | 31.6 (16.9-55.7) | |
| Range | 1.2-426.9 | 10.0-344.4 | 1.2-426.9 | <0.0001^{‡} |
| Number of cigarettes per day | | | | |
| N | 2237 | 113 | 2350 | |
| Mean (SD) | 24.68 (10.51) | 24.84 (12.51) | 24.69 (10.61) | |
| Median (IQR) | 25 (20-25) | 25 (20-25) | 25 (20-25) | |
| Range | 1-100 | 5-100 | 1-100 | 0.8943^{‡} |
| Smoking duration | | | | |
| N | 2235 | 112 | 2347 | |
| Mean (SD) | 43.97 (5.87) | 45.91 (6.06) | 44.06 (5.89) | |
| Median (IQR) | 44 (40-48) | 46.5 (42-50) | 44 (40-48) | |
| Range | 11-69 | 27-60 | 11-69 | 0.0012^{‡} |
| Quit time | | | | |
| N | 2237 | 113 | 2350 | |
| Mean (SD) | 17.85 (21.29) | 17.62 (21.71) | 17.84 (21.31) | |
| Median (IQR) | 0 (0-41) | 05 (0-41) | 0 (0-41) | |
| Range | 0-61 | 0-55 | 0-61 | 0.9128^{‡} |

Abbreviations: FEV₁% pred, forced expiratory volume in 1 second % predicted; ng/ml, nanograms per milliliter; SD, standard deviation; IQR, inter-quartile range.
*Fisher exact test p-value
^{†}Non-parametric p-value
^{‡}Satterthwaite's unequal variance t-test. P-value

The Carotene and Retinol Efficacy Trial (CARET) Study

The validation test samples were comprised of sera collected from participants of the Carotene and Retinol Efficacy Trial (CARET). CARET was a multicenter, randomized, double-blind, placebo-controlled study to evaluate the efficacy and safety of daily supplementation of 30 mg β-carotene and 25,000 IU retinyl palmitate on primary lung cancer prevention.¹¹ Eligible participants were either (a) men and women aged 50 to 69 years who were either current or former smokers (quit within previous 6 years) and had at least 20 pack-years of cigarette smoking (N=14,254) or (b) men, 45-69 years of age who were current or former smokers (quit no more than 15 years prior) and had a substantial history of occupational asbestos exposure (N=4,060). Participants were enrolled from 1985 to 1994 and followed for cancer and mortality outcomes until 2005. For the present study, we randomly selected 61 current smokers who developed NSCLC during follow-up and analyzed pro-SFTPB in serum samples, which had been collected within 12 months prior to the diagnosis of NSCLC. For each case, two controls, who were free of lung cancer throughout CARET follow-up, were selected, matched for age, gender, smoking history (current smoker), study enrollment cohort, and the date of blood draw. One to two case-control matching was possible for all cases except for one subject, leading to 121 control subjects. The clinical characteristics of the CARET participants are provided in Table 2.

TABLE 2
Clinical characteristics of subjects in the CARET set

| | Controls (%) | Cases (%) | P Value |
|---|---|---|---|
| Total | 121 (100) | 61 (100) | |
| Age (years) | | | |
| Mean | 64.1 | 64.3 | 0.8254 |
| SD | 6.3 | 5.9 | |
| Gender | | | |
| Female | 32 (25.1) | 16 (26.2) | 1 |
| Male | 89 (74.9) | 45 (73.8) | |
| Smoking (pack-year) | | | |
| Mean | 46.9 | 58.7 | <0.0001 |
| SD | 16.9 | 22.0 | |
| Asbestos exposure | | | |
| Yes | 26 (21.5) | 13 (21.3) | 1 |
| No | 95 (78.5) | 48 (78.7) | |
| BMI (kg/m²)* | | | |
| Mean | 26.7 | 26.8 | 0.8583 |
| SD | 5.2 | 5.7 | |
| Stage | | | |
| I and II | — | 11 (18.0) | |
| III and IV | — | 40 (65.6) | |
| Unknown | — | 10 (16.4) | |
| Histology | | | |
| Adenocarcinoma | — | 26 (42.6) | |
| Squamous | — | 17 (27.9) | |
| Other NSCLC | — | 18 (29.5) | |
| Time span to diagnosis (months) | | | |
| Mean | — | 6.2 | |
| Range | — | 0.9-12.4 | |

*BMI data for one control subject was not available.

All serum samples were obtained following informed consent and with Institutional Review Board approval of Fred Hutchinson Cancer Research Center.

Pro-SFTPB Assay

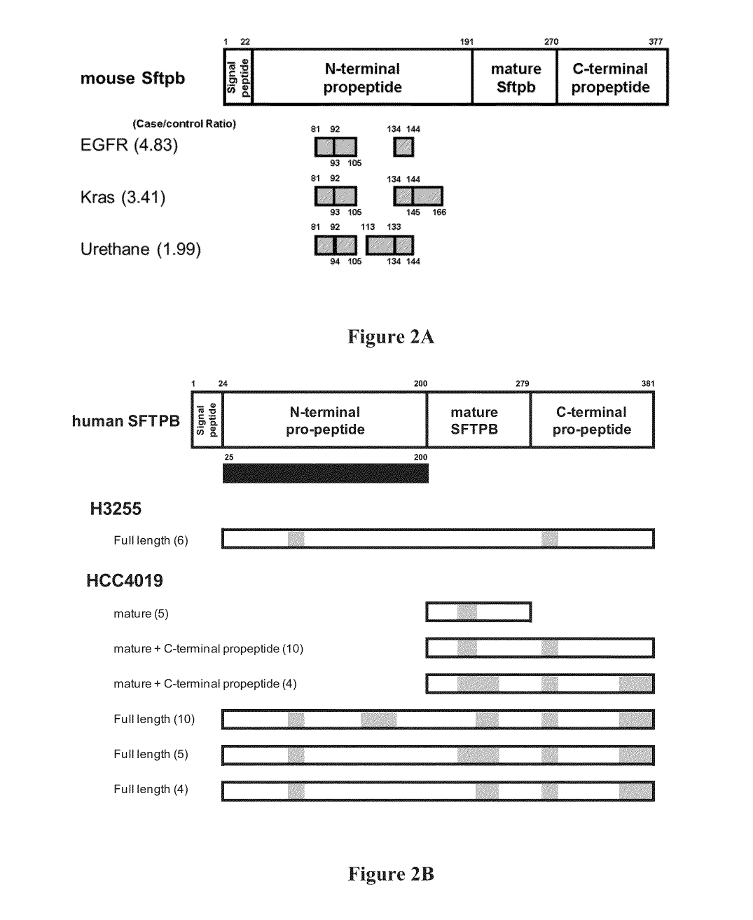

Using mass spectrometry, we determined the presence of N-terminal and C-terminal pro-peptides of SFTPB in circulation of mice harboring lung adenocarcinoma and in the conditioned media of NSCLC cell lines (FIGS. 2A and 2B).

Mouse monoclonal antibodies against the N-terminus of pro-SFTPB (FIG. 2B) were raised, leading to the development of a sandwich pro-SFTPB ELISA which specifically reacted with pro-SFTPB and did not react with other surfactant proteins or mature SFTPB, as follows.

Production of Recombinant Pro-SFTPB Protein

Sequence of pulmonary surfactant-associated protein B (SFTPB) was taken from UniProtKB/Swiss-Prot entry P07988. DNA coding the N-terminal pro-peptide (25-200 aa) was synthesized and then optimized using GeneArt (Regensburg, Germany). The synthesized gene was ligated with the pDONR221 vector (Invitrogen, Darmstadt, Germany) and subcloned into pDESTVH8G (modified pTT5V5H8 plasmid from Biotechnology Research Institute, National Research Council Canada, Montreal). After sequence confirmation, plasmid DNA was prepared and transfected into HEK293-EBNA1 cells in suspension with linear PEI for production of recombinant protein.¹² The resultant cell culture medium was clarified by centrifugation (13,000 rpm, 1 hour, 4° C.) and filtration (0.45μ), and bound to Ni2+-NTA resin (25 ml of a 50% slurry, pre-equilibrated in MEB) in batch mode and packed into a chromatographic column connected to an AKTA purifier. The column was washed extensively with MEB to replace 6 M GuHCl with 8 M urea, and eluted using a step imidazole gradient in 8M urea-MEB. Column fractions containing purified protein, based on SDS-PAGE analysis, were pooled and dialyzed against 20 mM Tris-HCl (pH 8.5) buffer containing 50 mM NaCl. The purified protein preps was analyzed by SDS-PAGE and western blotting, using penta-His mAb, in conjunction with anti-mouse IgG-HRPO conjugate and subsequently confirmed using mass spectrometry.

Pro-SFTPB ELISA Assay

SFTPB-specific monoclonal antibodies (mAb), as shown in Table 3, and a sandwich ELISA were developed by the Antibody Research Unit of the BC Cancer Agency in Victoria, BC.

TABLE 3
Description of murine monoclonal antibody
hybridomas specific for NT pro-SFTPB

| Item Name | Description |
|---|---|
| ACcSFTPB.3409 | Antibody Hybridoma (477 antibody) |
| ACcSFTPB.3473 | Antibody Hybridoma (515 antibody) |

The standards were calibrated according to the absolute mass of the recombinant antigen, as follows. Costar white high binding 96 well plate (Corning, Corning, N.Y.) were coated with 100 μl/well of 1.00 μg/ml purified mAb515 in 0.1M carbonate buffer (33.5 mM Na₂CO₃, 0.1 M NaHCO₃, pH 9.6) and incubated overnight at 4° C. Plasma samples with 1:100 dilution and various amounts of N-terminal pro-peptide of SFTPB as standards were added to the wells. Plates were blocked with to 200 μl/well of Superblock (Pierce, Rockford, Ill.) and incubated at room temperature (RT) for 2.5 hours. Plates were washed with a protocol including six wash steps in TBS/0.1% Tween-20 (TBST) using a Skanwasher plate washer (Molecular Devices, Union City, Calif.). Patient serum, control serum or pancreatic juice was diluted 1:10 in 1× Reagent Diluent (R&D Systems, Minneapolis, Minn.) and incubated for 2 hours at RT on a shaker. All samples and controls were assayed in duplicate. Plates were washed and incubated with 100 μl per well of 0.5 μg/ml biotinylated mAb477 in TBST for 2 hours at RT with shaking. Plates were washed and incubated with 100 μl per well streptavidin-alkaline phosphatase conjugate (Applied Biosystems Inc, Foster City, Calif.) at 1:2500 in TBST for 1 hour on a shaker at RT. After washing, the plates were incubated with 100 μl/well of 0.4 mM chemiluminescent CSPD® Substrate with Emerald-II™ Enhancer (Applied Biosystems) at RT for 20 min in dark and read on an EnVision multilabel plate reader (PerkinElmer, Waltham, Mass.) and analyzed using Envision software 1.12.

We then validated this assay with plasma samples obtained at the time of diagnosis from subjects with operable NSCLC (n=28) and healthy controls (n=38). These samples had previously been analyzed for levels of mature SFTPB by ELISA (Table 4).

TABLE 4
Demographics of newly diagnosed NSCLC set.

| | Control (%) | NSCLC (%) |
|---|---|---|
| Total | 38 (100) | 28 (100) |
| Age (years) | | |
| Mean | 61.9 | 63.3 |
| SD* | 9.7 | 10.7 |
| Gender | | |
| Female | 21 (55.3) | 13 (46.4) |
| Male | 17 (44.7) | 15 (53.6) |
| Histology | | |
| Adenocarcinoma | — | 17 (60.7) |
| Squamous | — | 11 (39.3) |

*SD, standard deviation.

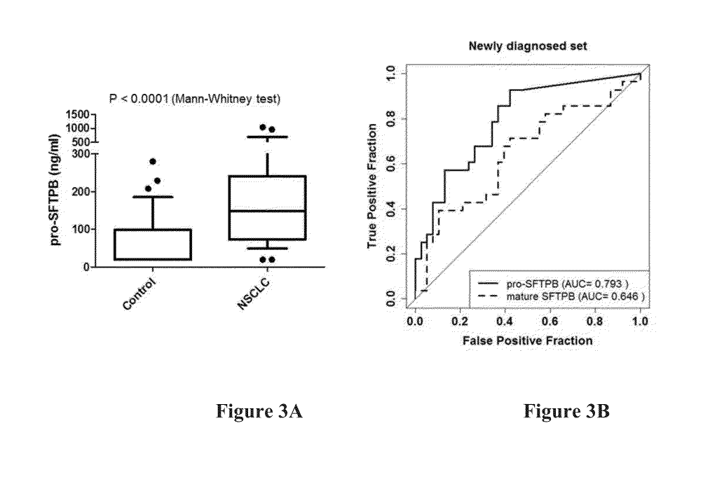

Plasma levels of pro-SFTPB were significantly higher in cases compared to controls (P<0.0001 by Mann-Whitney test) (FIG. 3A). The AUC of pro-SFTPB was superior to that of mature SFTPB⁷ (0.793 and 0.646, respectively; FIG. 3B).

For the PanCan study, the baseline plasma samples (i.e., samples taken at the time of enrollment) were used for the assay. For both the PanCan and CARET studies, samples were blinded and analyzed using anti-pro-SFTPB mouse monoclonal antibodies. All samples were assayed in duplicate. Anti-pro-SFTPB mouse monoclonal antibody (#464) was biotinylated with EZ-Link® Sulfo-NHS-LC-Biotin (Thermo Scientific) and used for incubation at 0.5 μg/ml. After washing, each well was incubated with Streptavidin-horseradish peroxidase followed by incubation of color reagents and adding stop solution (R&D Systems). The absorbance was measured at 450 nm with a SpectraMax M5 microplate reader (Molecular Devices) or with a Versamax microplate reader (Molecular Devices). For samples whose pro-SFTPB levels were below the level of detection, we assigned a value that was one-half of the detection limit. The median coefficient of variation was 6.1%. Because the PanCan Study and the CARET Study used different standards, the absolute levels of pro-SFTPB between the studies are not directly comparable.

Statistical Methods

Descriptive comparisons of study variables between groups used Fisher's exact test for categorical data, t-test for continuous data and nonparametric test of trend for ordinal data. Multivariable logistic regression models were used to evaluate whether pro-SFTPB was independently associated with lung cancer. Known risk factors for lung cancer were evaluated in models, and included age, sex, body mass index (BMI), personal history of cancer, family history of lung cancer, forced expiratory volume in 1 second percent predicted (FEV₁% pred), average number of cigarettes smoked per day, and duration smoked. Pro-SFTPB was right skewed and in modeling log-transformed pro-SFTPB (log-proSFTPB) was used. Selected interaction terms were evaluated including main effects and cross-product terms in the model and nonlinear associations between continuous variables and lung cancer were evaluated by multivariable fractional polynomials.¹³ No interactions or nonlinear relationships were found to be significant.

Regarding prediction, improvement in discrimination was assessed by comparing receiver operator characteristics area under the curves (AUC) between nested models with and without log-proSFTPB. For AUCs, 95% confidence intervals (95% CI) were prepared using bootstrap resampling with 1000 samples.¹⁴ Calibration was assessed by evaluating the mean and 90^{th} percentile absolute errors.¹⁵ For each model, we calculated a Brier score.¹⁶ Optimism or overfit in models was assessed using bootstrap method by applying Harrell's RMS package in R (version 3.0.1).^{15,17} Bootstrap-optimism-corrected estimates of AUCs and Brier statistics are also presented. For comparative purposes we produced Cox proportional hazards survival models analogous to our logistic regression models. All analyses, statistics and figures were prepared using Stata 12.1MP (StataCorp, College Station, Tex.). All presented p-values are two-sided.

In the CARET study, pro-SFTPB levels were categorized into quintiles based on the distribution in control subjects. Logistic regression was performed to obtain odds ratio and adjusted odds ratios were generated using multiple logistic regression analyses in which we controlled for matching variables (age, gender, smoking status, enrollment period, and blood draw visit), pack-years, years since quitting smoking, asbestos exposure, and BMI.

Results

Pro-SFTPB levels were measured in 2,485 individuals, who enrolled in the multicenter Pan-Canadian Early Detection of Lung Cancer (PanCan) Study (ClinicalTrials[dot]gov NCT00751660), using plasma samples collected at the baseline visit. Multivariable logistic regression models were used to evaluate the predictive ability of pro-SFTPB in addition to known lung cancer risk factors. Calibration and discrimination were evaluated, the latter by an area under the receiver operator characteristics curve (AUC). External validation was performed with samples collected in the Carotene and Retinol Efficacy Trial (CARET) participants using a case-control study design.

Study Populations

PanCan Study

Pro-SFTPB data were available for 2,485 individuals. The minimum, median and maximum follow-up durations were 0.02, 3.02 and 4.36 years. During this follow-up period, 187 (7.4%) individuals were lost to follow-up. Loss-to-follow-up status was not associated with pro-SFTPB (p=0.527), nor were pro-SFTPB levels associated with time to loss-to-follow-up (p=0.954).

Pro-SFTPB was measured in nanograms per milliliter (ng/ml) and for pro-SFTPB the mean (standard deviation, SD) and median (interquartile range, IQR) were 45.32 (SD 44.64) and 31.93 (IQR 16.92-56.26), respectively. Distributions of pro-SFTPB by study variables are presented in Table 5.

TABLE 5
Distribution of pro-SFTPB by selected study various variables

| Variables | N | Mean (SD) | Median (IQR) | Range | P-value* |
|---|---|---|---|---|---|
| Pro-SFTPB (ng/ml) | 2485 | 45.32 (44.64) | 31.93 (16.92-56.26) | 1.15-426.86 | NA |
| log(proSFTPB) | 2485 | 3.49 (0.83) | 3.49 (2.89-4.05) | 0.767-6.059 | NA |
| Age (years) | | | | | |
| 50-54 | 270 | 46.26 (47.19) | 33.27 (16.21-57.36) | 1.15-324.35 | |
| 55-59 | 491 | 45.34 (44.13) | 33.11 (17.28-57.67) | 2.56-426.86 | |
| 60-64 | 791 | 42.82 (42.81) | 29.82 (16.26-53.85) | 1.42-330.24 | |
| 65-69 | 642 | 44.10 (43.05) | 30.18 (16.72-54.64) | 2.85-344.38 | |
| ≧70 | 291 | 53.93 (50.30) | 37.98 (19.12-66.82) | 4.29-294.48 | 0.219^{†} |
| Sex | | | | | |
| Male | 1377 | 46.73 (45.54) | 33.24 (17.59-58.35) | 1.42-426.86 | |
| Female | 1108 | 43.58 (43.47) | 30.02 (16.31-53.45) | 1.15-344.38 | 0.0794 |
| Body Mass Index (kg/m²) | | | | | |
| Underweight (<18.5) | 27 | 37.28 (24.57) | 28.76 (18.95-55.37) | 7.45-98.90 | |
| Normal (18.5-24.9) | 919 | 47.70 (46.39) | 33.70 (18.63-57.67) | 1.15-426.86 | |
| Overweight (25-29.9) | 1123 | 45.53 (45.17) | 31.64 (16.01-58.68) | 2.18-372.25 | |
| Obese (≧30) | 416 | 40.03 (39.62) | 28.32 (15.87-48.71) | 1.42-282.03 | 0.001^{†} |
| Personal History of Cancer | | | | | |
| No | 2321 | 45.14 (44.75) | 31.65 (16.88-55.85) | 1.15-426.86 | |
| Yes | 156 | 47.95 (43.88) | 34.90 (18.58-59.51) | 2.18-282.03 | 0.4399 |
| Family History of Cancer | | | | | |
| No | 1625 | 45.90 (45.13) | 32.08 (17.29-56.94) | 1.42-426.86 | |
| Yes | 819 | 43.71 (42.59) | 30.85 (16.21-55.79) | 1.15-330.24 | 0.2389 |
| Pneumonia | | | | | |
| No | 1813 | 44.29 (43.32) | 31.79 (16.88-55.40) | 1.42-426.86 | |
| Yes | 665 | 48.22 (48.15) | 31.73 (16.96-58.71) | 1.15-324.35 | 0.0651 |
| Emphysema | | | | | |
| No | 2315 | 44.82 (43.73) | 31.71 (16.94-55.80) | 1.15-426.86 | |
| Yes | 160 | 51.97 (53.19) | 36.25 (15.38-64.30) | 3.14-328.88 | 0.0984 |
| Smoking status | | | | | |
| Former smoker | 938 | 35.51 (42.42) | 21.26 (12.13-40.81) | 1.15-426.86 | |
| Current smoker | 1547 | 51.28 (44.92) | 38.48 (21.77-63.99) | 2.18-344.38 | <0.0001 |
| Race/Ethnicity | | | | | |
| White | 2410 | 45.50 (44.95) | 31.96 (16.93-56.33) | 1.15-426.86 | |
| Asian | 29 | 40.28 (34.98) | 33.30 (15.62-50.70) | 5.04-160.48 | |
| Aboriginal | 8 | 35.19 (26.60) | 30.89 (17.19-47.46) | 3.92-86.55 | |
| Black or African Canadian | 13 | 45.89 (45.79) | 27.05 (18.63-64.41) | 5.90-172.51 | |
| Other | 15 | 33.53 (23.13) | 30.94 (11.95-41.49) | 6.15-78.02 | 0.396^{†} |
| Education | | | | | |
| 8^{th} grade or less | 74 | 56.22 (42.43) | 47.60 (23.77-75.39) | 7.22-241.07 | |
| 9^{th} to 11^{th} grade | 326 | 53.33 (51.04) | 38.23 (18.48-67.05) | 1.15-372.25 | |
| High school graduate | 649 | 42.99 (43.20) | 29.96 (16.68-52.69) | 1.42-344.38 | |
| Technical/Vocational | 264 | 48.74 (48.06) | 33.62 (17.46-64.93) | 2.15-330.24 | |
| Associate degree/some college | 475 | 43.66 (39.07) | 32.03 (16.68-57.59) | 2.39-274.28 | |
| Bachelor's degree | 426 | 42.99 (46.71) | 28.99 (15.65-51.78) | 4.15-426.86 | |
| Advanced degree | 271 | 41.57 (41.32) | 26.62 (16.54-52.06) | 2.18-235.45 | <0.001^{†} |

Abbreviations: IQR = Inter-quartile range;
NA, not applicable;
ng/ml, nanograms/milliliter;
SD = Standard deviation;
^{†}Non-parametric P-value;
*P-values are for Satterthwaite's unequal variance t-test, unless otherwise marked:
^{†}analysis of variance.(do EDLC-PCS-do/PanCanBiomarkerTables_20FEB2013.do)

Prediction Models

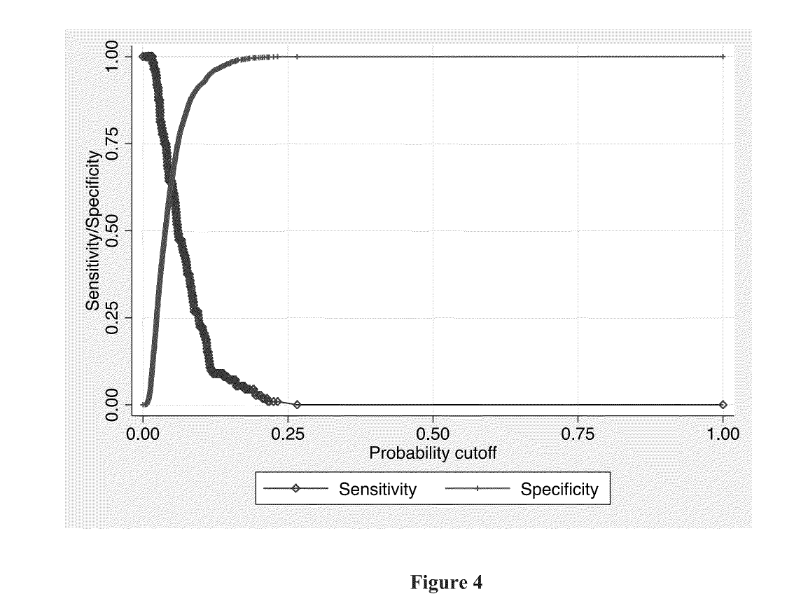

In an unadjusted logistic model of log-proSFTPB predicting lung cancer, the odds ratio was 2.331 (95% CI 1.837-2.958; p<0.001) and the AUC was 0.690 (95% CI 0.642-0.735). The sensitivity and specificity for log-proSFTPB over the range of model probabilities are presented in FIG. 4. When the aforementioned model probability for positivity is set to p>0.032, sensitivity is 80.4%, specificity is 40.1%, the positive predictive value is 6.4% and the negative predictive value is 97.6%. In the unadjusted logistic model of log-proSFTPB for events occurring at least one year following baseline blood draw, AUC was 0.655 (95% CI 0.570-0.719).

In the logistic model fully adjusted for lung cancer risk factors including smoking and non-smoking predictors, log-proSFTPB was a significant independent predictor of lung cancer (OR=2.220, 95% CI 1.727-2.853; p<0.001) (Table 6).

TABLE 6
Logistic regression prediction model with log transformed pro-SFTPB predicting
lung cancer in the Pan-Canadian Early Detection of Lung Cancer Study (N = 2,233)

| Predictor Variables | Odds ratio (95% CI; p-value) | Beta coefficients |
|---|---|---|
| Age (per year) | 1.023 (0.978-1.070; p = 0.326) | .0226304 |
| Sex (male vs. female) | 0.592 (0.391-0.897; p = 0.013) | −.5239236 |
| Body mass index (kilograms/meter²) | 0.957 (0.912-1.005; p = 0.077) | −.0439466 |
| Personal history of cancer (yes vs. no) | 1.379 (0.684-2.780; p = 0.369) | .3215962 |
| Pneumonia (yes vs. no) | 1.341 (0.876-2.055; p = 0.177) | .2936113 |
| Family history of cancer (yes vs. no) | 1.412 (0.923-2.160; p = 0.112) | .344805 |
| FEV₁ % predicted | 0.270 (0.091-0.804; p = 0.019) | −1.310217 |
| Cigarette smoked per day | 1.010 (0.991-1.030; p = 0.292) | .0102629 |
| Smoking duration | 1.034 (0.989-1.082; p = 0.142) | .0336636 |
| Log(proSFTPB) | 2.220 (1.727-2.853; p < 0.001) | .7975728 |
| Model constant | | −6.948646 |

Abbreviations: CI, confidence interval;
N, number

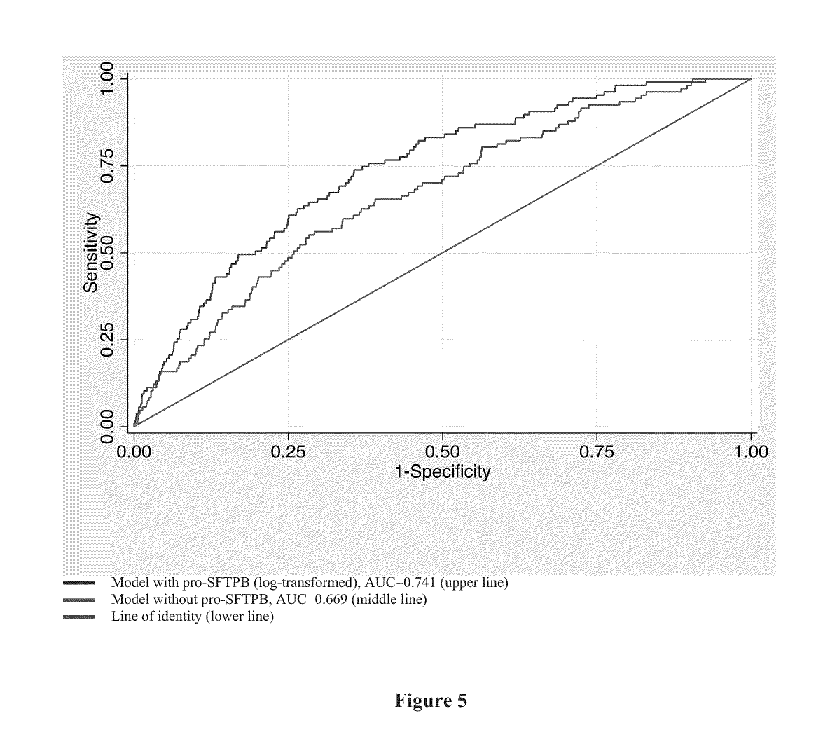

In the fully adjusted model, when the analysis was limited to lung cancers occurring within the first year, the OR for proSFTPB was 2.53 (95% CI 1.79-3.59; p<0.001). The AUCs for the full logistic models with and without log-proSFTPB were 0.741 (95% CI 0.696-0.783) and 0.669 (95% CI 0.620-0.717) (p-value for difference in AUC=0.0007) (FIG. 5). The respective bootstrap-bias-corrected AUCs were 0.718 and 0.636. When proSFTPB concentrations were grouped into quintiles, the univariate OR per 1 level change was 1.62 (95% CI 1.39-1.89; p<0.001) with a model AUC of 0.579 (95% CI 0.526-0.626) and in the multivariable model, OR was 1.59 (95% CI 1.36-1.87; p<0.001) with a model AUC of 0.730 (95% CI 0.680-0.775). This improvement in discrimination attributable to pro-SFTPB is large in magnitude compared to most lung cancer predictors.¹⁸ Of the 113 lung cancers, 96 (85.0%) were stage I or II. When the full model was estimated in these individuals, log-proSFTPB remained a statistically significant predictor (OR=2.195, 1.679-2.870; p<0.001), and significantly improved the AUC compared to the nested model excluding log-proSFTPB (0.735 vs. 0.659, p=0.0007).

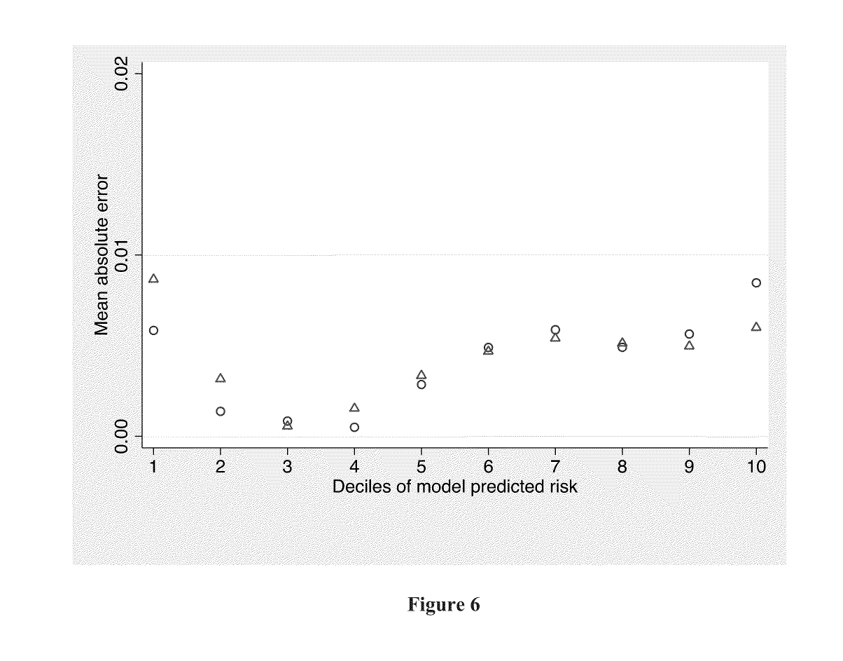

The mean and 90^{th} percentile absolute error (observed minus predicted probabilities) in the model without log-proSFTPB were 0.005 and 0.007, and for the model with log-proSFTPB were 0.004 and 0.010. For both models the mean absolute errors in all deciles of model predicted risk were less than 1% (FIG. 6). For the full model with versus the nested model without log-proSFTPB, the Brier scores were 0.0438 and 0.0448 and the bootstrap-bias-corrected scores were 0.0442 and 0.0450, respectively. These statistics indicate that calibration was excellent in both models but slightly better in the model with log-proSFTPB.

The magnitude of Cox model hazard ratios and confidence intervals were similar to the odds ratios in the logistic models (Table 7).

TABLE 7
Logistic and Cox proportional hazards regression models including
pro-SFTPB predicting the outcome lung cancer in the PanCan Study

| | Odds ratio (95% CI; p-value) | Hazard ratio (95% CI; p-value) |
|---|---|---|
| Predictor Variables | N = 2233 | N = 2411 |
| Age (per year) | 1.023 (0.978-1.070; p = 0.326) | 1.022 (0.979-1.066; p = 0.329) |
| Sex (female vs. male) | 0.592 (0.391-0.897; p = 0.013) | 0.609 (0.410-0.906; p = 0.014) |
| Body mass index | 0.957 (0.912-1.005; p = 0.077) | 0.964 (0.921-1.010; p = 0.123) |
| (kilograms/meter²) | | |
| Personal history of cancer | 1.379 (0.684-2.780; p = 0.369) | 1.384 (0.719-2.664; p = 0.331) |
| (yes vs. no) | | |
| Pneumonia (yes vs. no) | 1.341 (0.876-2.055; p = 0.177) | 1.276 (0.849-1.919; p = 0.241) |
| Family history of cancer (yes no) | 1.412 (0.923-2.160; p = 0.112) | 1.354 (0.906-2.023; p = 0.140) |
| FEV₁% predicted | 0.270 (0.091-0.804; p = 0.019) | 0.320 (0.113-0.908; p = 0.032) |
| Cigarettes smoked per day | 1.010 (0.991-1.030; p = 0.292) | 1.009 (0.991-1.027; p = 0.317) |
| Smoking duration (in years) | 1.034 (0.989-1.082; p = 0.142) | 1.033 (0.990-1.079; p = 0.138) |
| Log(proSFTPB) | 2.220 (1.727-2.853; p < 0.001) | 2.115 (1.674-2.671; p < 0.001) |
| Performance statistics | AUC = .741 | C-statistic = .734 |

Abbreviations: CI, confidence interval;
N, number;

When the full Cox model was limited to lung cancers which were diagnosed >1 year and >2 years after study entry, the hazard ratios for log-proSFTPB were 1.875 (95% CI 1.346-2.610; p<0.001; event number=53), and 1.650 (95% CI 1.028-2.649; p=0.038; event number=26).

CARET Study

Our sample size and number of outcome events were adequate to find statistically significant results regarding the relationship between plasma levels of pro-SFTPB and lung cancer risk, providing effect estimates with precise confidence intervals, and demonstrating significant incremental improvement in prediction. However, because over 75% of the lung cancer cases diagnosed in the Pan Can Study were adenocarcinomas, we could not adequately evaluate whether the relationship between pro-SFTPB and lung cancer risk differed across different histological tumor sub-types. In the CARET study, which proportionately had more cases of squamous cell carcinoma, pro-SFTPB appeared to be less predictive in squamous cell carcinomas than with adenocarcionomas (Table 8).

TABLE 8
Serum pro-SFTPB levels and sample characteristics in the CARET set.

| | Matched Control | | NSCLC | | | | |
|---|---|---|---|---|---|---|---|
| | | proSFTPB | | proSFTPB | | | |
| | | (Median | | (Median | | ROC analysis | |
| | | (IQR)) | | (IQR)) | | | 95% |
| | N | (ng/ml) | N | (ng/ml) | P value | AUC | CI |
| Total | 121 | 201.7 | 61 | 286.3 | <0.0001 | 0.683 | 0.604- |
| | | (113.0- | | (213.8- | | | 0.761 |
| | | 316.2) | | 348.7) | | | |
| Gender | | | | | | | |
| Female | 32 | 164.6 | 16 | 252.0 | 0.0259 | 0.699 | 0.552- |
| | | (19.50- | | (192.1- | | | 0.847 |
| | | 227.1) | | 323.0) | | | |
| Male | 89 | 215.7 | 45 | 292.7 | 0.0004 | 0.686 | 0.593- |
| | | (141.7- | | (225.2- | | | 0.779 |
| | | 323.2) | | 354.4) | | | |
| Asbestos | | | | | | | |
| exposure | | | | | | | |
| Yes | 26 | 213.4 | 13 | 351.8 | 0.0018 | 0.811 | 0.675- |
| | | (147.5- | | (261.4- | | | 0.946 |
| | | 337.3) | | 741.0) | | | |
| No | 95 | 198.2 | 48 | 278.2 | 0.0034 | 0.650 | 0.559- |
| | | (107.0- | | (199.8- | | | 0.741 |
| | | 304.6) | | 329.3) | | | |
| Stage | | | | | | | |
| I and II | 21 | 219.1 | 11 | 216.3 | 0.4501 | 0.584 | 0.385- |
| | | (79.20- | | (198.1- | | | 0.784 |
| | | 295.2) | | 290.1) | | | |
| III and IV | 80 | 202.9 | 40 | 301.3 | 0.0004 | 0.700 | 0.604- |
| | | (127.6- | | (235.2- | | | 0.796 |
| | | 325.7) | | 396.3) | | | |
| Histology | | | | | | | |
| Adeno- | 52 | 183.8 | 26 | 283.5 | 0.0126 | 0.674 | 0.547- |
| carcinoma | | (77.55- | | (184.4- | | | 0.801 |
| | | 296.2) | | 370.0) | | | |
| Squamous | 34 | 212.6 | 17 | 315.5 | 0.1364 | 0.630 | 0.479- |
| | | (115.0- | | (231.3- | | | 0.781 |
| | | 385.1) | | 339.6) | | | |
| Other | 35 | 214.9 | 18 | 292.5 | 0.0132 | 0.710 | 0.563- |
| NSCLC | | (97.10- | | (208.2- | | | 0.858 |
| | | 300.3) | | 409.4) | | | |
| Time | | | | | | | |
| span to | | | | | | | |
| diagnosis | | | | | | | |
| 0-6 | 61 | 205.0 | 31 | 292.7 | 0.0027 | 0.692 | 0.585- |
| months | | (144.4- | | (216.3- | | | 0.800 |
| | | 319.4) | | 351.0) | | | |
| ≧6 months | 60 | 194.7 | 30 | 286.0 | 0.0066 | 0.676 | 0.561- |
| | | (72.14- | | (195.5- | | | 0.791 |
| | | 300.1) | | 340.9) | | | |

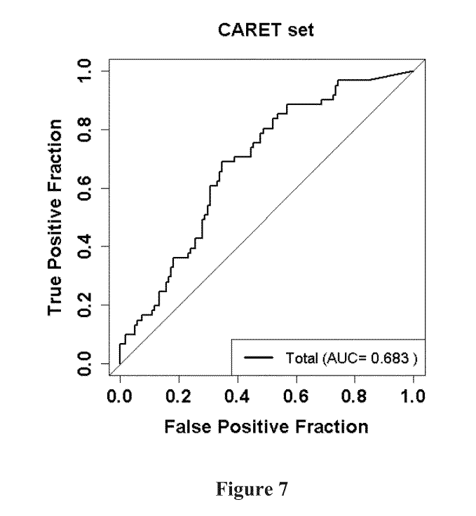

Pro-SFTPB levels were significantly higher among NSCLC cases compared with controls (P<0.0001) and ROC analysis yielded AUC of 0.683 (95% CI, 0.604-0.761) (Table 9 and FIG. 7).

TABLE 9
Relationship Between Pro-SFTPB and the Risk
of NSCLC in the CARET Study According To Quartiles
of Serum Pro-SFTPB Concentrations.

| | Q1 | Q2 | Q3 | Q4 | Q5 | P_{trend} |
|---|---|---|---|---|---|---|
| Total subjects | 26 | 29 | 35 | 45 | 46 | |
| (No.) | | | | | | |
| Control (No.)* | 24 | 24 | 24 | 24 | 24 | |
| NSCLC (No.) | 2 | 5 | 11 | 21 | 22 | |
| Odds Ratio† | 1 | 2.64 | 5.61 | 11.63 | 12.34 | 0.0002 |
| (95% CI) | (Refer- | (0.46- | (1.11- | (2.40- | (2.54- | |
| | ence) | 15.11) | 28.37) | 56.33) | 60.00) | |
| Adjusted Odds | 1 | 2.74 | 6.66 | 12.24 | 9.64 | 0.001 |
| Ratio‡ | | | | | | |
| (95% CI) | (Refer- | (0.42- | (1.15- | (2.22- | (1.74- | |
| | ence) | 17.12) | 38.32) | 67.43) | 53.34) | |

*one sample was excluded from this analysis due to missing BMI data.
†adjusted for matching variables (age, gender, smoking status, enrollment period, and blood draw visit).
‡adjusted for matching variables, pack-years, years since quitting smoking, asbestos exposure, and BMI.
Abbreviations: CARET, Carotene and Retinol Efficacy Trial; CI, confidence interval; NSCLC, non-small cell lung cancer; Q, quartile

In terms of histological subgroups, pro-SFTPB levels were significantly elevated in adenocarcinoma, but not in squamous cell carcinoma compared with matched controls. In multivariate logistic regression analysis, the risk of NSCLC increased along with the pro-SFTPB concentration gradient in the CARET set (P_{trend}=0.0002, adjusted for matching variables; Table 9). The risk of NSCLC also increased per quintile increase (OR=1.77, 95% CI=1.35-2.33, adjusted for matching variables; OR=1.64, 95% CI=1.22-2.20, adjusted for matching variables, pack-years, years since quitting smoking, asbestos exposure, and BMI).

The results indicate that plasma pro-SFTPB is significantly and independently associated with lung cancer and is an independent predictor of lung cancer. Furthermore, pro-SFTPB was associated with early stage (I and II) lung cancer and with lung cancers diagnosed >1 year after plasma collection.

### REFERENCES

1. Siegel R, Naishadham D, Jemal A: Cancer statistics, 2012. CA Cancer J Clin 62:10-29, 2012
2. National Lung Screening Trial Research T, Aberle D R, Adams A M, et al: Reduced lung-cancer mortality with low-dose computed tomographic screening. N Engl J Med 365:395-409, 2011
3. Goulart B H, Bensink M E, Mummy D G, et al: Lung cancer screening with low-dose computed tomography: costs, national expenditures, and cost-effectiveness. J Natl Compr Canc Netw 10:267-75, 2012
4. Guttentag S, Robinson L, Zhang P, et al: Cysteine protease activity is required for surfactant protein B processing and lamellar body genesis. Am J Respir Cell Mol Biol 28:69-79, 2003
5. Khoor A, Whitsett J A, Stahlman M T, et al: Utility of surfactant protein B precursor and thyroid transcription factor 1 in differentiating adenocarcinoma of the lung from malignant mesothelioma. Hum Pathol 30:695-700, 1999
6. O'Reilly M A, Gazdar A F, Clark J C, et al: Glucocorticoids regulate surfactant protein synthesis in a pulmonary adenocarcinoma cell line. Am J Physiol 257:L385-92, 1989
7. Taguchi A, Politi K, Pitteri S J, et al: Lung cancer signatures in plasma based on proteome profiling of mouse tumor models. Cancer Cell 20:289-99, 2011
8. Tammemagi C M, Pinsky P F, Caporaso N E, et al: Lung cancer risk prediction: prostate, lung, colorectal and ovarian cancer screening trial models and validation. Journal of the National Cancer Institute 103:1058-68, 2011
9. Tammemagi M, Hormuzd K, Hocking W, et al: Selection Criteria for Lung-Cancer Screening. New England Journal of Medicine 368:728-736, 2013
10. Miller M R, Hankinson J, Brusasco V, et al: Standardisation of spirometry. Eur Respir J 26:319-38, 2005
11. Goodman G E, Thornquist M D, Balmes J, et al: The Beta-Carotene and Retinol Efficacy Trial: incidence of lung cancer and cardiovascular disease mortality during 6-year follow-up after stopping beta-carotene and retinol supplements. J Natl Cancer Inst 96:1743-50, 2004
12. Toni R, Bisson L, Durocher Y. 2008. Transfection of HEK293-ERNA1 cells in suspension with linear PEI for production of recombinant proteins. CSH Protoc. 2008:pdb.prot4977. doi:10.1101/pdb.prot4977.
13. Royston P, Sauerbrei W: Multivariable model-building: a pragmatic approach to regression analysis based on fractional polynomials for modelling continuous variables. Chichester, West Sussex, England; Hoboken, N.J., John Wiley & Sons, 2008
14. Pepe M S, Longton G, Janes H: Estimation and comparison of receiver operating characteristic curves. The Stata Journal 9:1-16, 2009
15. Harrell F E: Regression modeling strategies: with applications to linear models, logistic regression, and survival analysis. New York, Springer, 2001
16. Ikeda M, Itoh S, Ishigaki T, et al: Application of resampling techniques to the statistical analysis of the Brier score. Methods Inf Med 40:259-64, 2001
17. Harrell F E: Regression Modeling Strategies: Package ‘rms’. 2010:1-217.
18. Lam S, Boyle P, Healey G F, et al: EarlyCDT-Lung: an immunobiomarker test as an aid to early detection of lung cancer. Cancer Prev Res (Phila) 4:1126-34, 2011.

All citations are hereby incorporated by reference.

The present invention has been described with regard to one or more embodiments. However, it will be apparent to persons skilled in the art that a number of variations and modifications can be made without departing from the scope of the invention as defined in the claims.

What is claimed is:

1. A monoclonal antibody, or an antigen-binding fragment thereof, that specifically binds the N-terminal propeptide of surfactant protein B (NT pro-SFTPB) or fragment thereof, or to a sequence substantially identical to the sequence of NT pro-SFTPB or fragment thereof.

2. The monoclonal antibody of claim 1 wherein the monoclonal antibody does not significantly bind one or more of mature surfactant protein B, the signal peptide of surfactant protein B, or the C-terminal propeptide of surfactant protein B.

3. The monoclonal antibody of claim 1 wherein the pro-SFTPB is human pro-SFTPB.

4. The monoclonal antibody of claim 1 wherein the NT pro-SFTPB consists essentially of the amino acid sequence as set forth in SEQ ID NO: 2, or a fragment thereof.

5. The monoclonal antibody of claim 1 wherein the monoclonal antibody is linked to a detectable label.

6. The monoclonal antibody of claim 5 wherein the detectable label is biotin.

7. The monoclonal antibody of claim 1 wherein the monoclonal antibody is linked to a solid support.

8. A hybridoma cell line producing the monoclonal antibody of claim 1.

9. The hybridoma cell line of claim 8 wherein the cell line is ACcSFTPB.3409 or ACcSFTPB.3473.

10. A composition comprising an antibody of claim 1, and at least one of a physiologically acceptable carrier, diluent, excipient, or stabilizer.

11. A method for detecting the N-terminal propeptide of surfactant protein B (NT pro-SFTPB) in a biological sample, the method comprising,
a) contacting the biological sample with the monoclonal antibody of claim 1 under conditions such that the antibody binds to the NT pro-SFTPB, if present in the biological sample; and
b) detecting the presence, absence, or amount of binding of the antibody to the NT pro-SFTPB from the biological sample.

12. The method of claim 11 wherein the monoclonal antibody is linked to a solid support.

13. The method of claim 12 wherein after the contacting, unbound components of the sample are washed away from the monoclonal antibody linked to the solid support while NT pro-SFTPB if present, remains bound to the monoclonal antibody, the method further comprising contacting the NT pro-SFTPB bound to the monoclonal antibody linked to the solid support with a second monoclonal antibody that binds NT pro-SFTPB and detecting the presence, absence, or amount of the second monoclonal antibody.

14. The method of claim 11 wherein the monoclonal antibody is linked to a detectable label.

15. The method of claim 11 wherein the biological sample is a biological fluid.

16. The method of claim 11 wherein the biological fluid is whole blood or plasma.

17. A kit comprising the monoclonal antibody of claim 1, together with instructions for detecting NT pro-SFTPB in a biological sample.

18. A method of diagnosing or prognosing lung cancer in a subject, the method comprising detecting the presence or absence of NT pro-SFTPB, wherein the presence of NT pro-SFTPB is a diagnosis or prognosis of lung cancer in the subject.

19. The method of claim 18 wherein the lung cancer is non-small cell lung cancer (NSCLC), lung adenocarcinoma or lung squamous cell carcinoma.

20. The method of claim 18 wherein the subject is a human.
